(19) United States
(12) Patent Application Publication
(10) Pub. No.: US 2007/0049588 A1
(43) Pub. Date: Mar. 1, 2007

(54) ATM INHIBITOR

(75) Inventors: Graeme Cameron Murray Smith, Cambridge (GB); Niall Morrison Barr Martin, Cambridge (GB); Andrew James Slade, Cambridge (GB); Keith Allan Menear, Cambridge (GB); Marc Geoffrey Hummersone, Cambridge (GB); Xiao-Ling Fan Cockcroft, Horsham (GB); Ian Timothy William Matthews, Horsham (GB); Laurent Jean Martin Rigoreau, London (GB); Roger John Griffin, Newcastle upon Tyne (GB); David Richard Newell, Newcastle upon tyne (GB); Nicola Jane Curtin, Newcastle upon Tyne (GB)

Correspondence Address: MICHAEL BEST & FRIEDRICH, LLP, ONE SOUTH PINCKNEY STREET, P O BOX 1806, MADISON, WI, 53701, US

(73) Assignee: KuDOS Pharmaceuticals Limited, Cambridge (GB)

(21) Appl. No.: 11/468,315

(22) Filed: Aug. 30, 2006

Related U.S. Application Data
(60) Provisional application No. 60/713,187, filed on Aug. 31, 2005.

(51) Int. Cl. A61K 31/5377; C07D 413/14

(52) U.S. Cl. 514/231.8; 544/79

(57) ABSTRACT

A compound of formula (I): 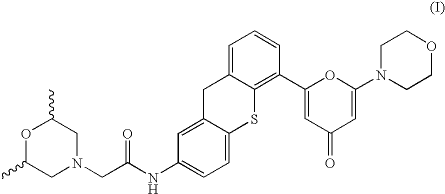 and isomers, salts, solvates, chemically protected forms, and prodrugs thereof, and their use in treating diseases ameliorated by the inhibition of ATM.

### CROSS-REFERENCE TO RELATED APPLICATIONS

This application claims priority to U.S. Provisional Application No. 60/713,187, filed Aug. 31, 2005, this application being incorporated herein by reference, in its entirety.

The present invention relates to a compound which acts as an ATM inhibitor, its use and synthesis.

Human DNA is constantly under attack from reactive oxygen intermediates principally from by-products of oxidative metabolism. Reactive oxygen species are capable of producing DNA single-strand breaks and, where two of these are generated in close proximity, DNA double strand breaks (DSBs). In addition, single- and double-strand breaks can be induced when a DNA replication fork encounters a damaged template, and are generated by exogenous agents such as ionising radiation (IR) and certain anti-cancer drugs (e.g. bleomycin, etoposide, camptothecin). DSBs also occur as intermediates in site-specific V(D)J recombination, a process that is critical for the generation of a functional vertebrate immune system. If DNA DSBs are left unrepaired or are repaired inaccurately, mutations and/or chromosomal aberrations are induced, which in turn may lead to cell death. To combat the serious threats posed by DNA DSBs, eukaryotic cells have evolved several mechanisms to mediate their repair. Critical to the process of DNA repair is the slowing down of cellular proliferation to allow time for the cell to repair the damage. A key protein in the detection of DNA DSBs and in the signalling of this information to the cell cycle machinery is the kinase ATM (ataxia telangiectasia mutated) (Durocher and Jackson, Curr Opin Cell Biol., 13, 225-31 (2001); Abraham, Genes Dev., 15, 2177-2196 (2001)).

The ATM protein is a ˜350 kDa polypeptide that is a member of the phosphatidylinositol (PI) 3-kinase family of proteins by virtue of a putative kinase domain in its carboxyl-terminal region (Savitsky, et al., Science, 268,1749-1753 (1995)). Classical PI 3-kinases, such as PI 3-kinase itself, are involved in signal transduction and phosphorylate inositol lipids that act as intracellular second messengers (reviewed in Toker and Cantley, Nature, 387, 673-676 (1997)). However, ATM bears most sequence similarity with a subset of the PI 3-kinase family that comprises proteins which, like ATM, are involved in cell cycle control and/or in the detection and signalling of DNA damage (Keith and Schreiber, Science, 270, 50-51 (1995); Zakian, Cell, 82, 685-687 (1995)). Notably there is no evidence to date that any members of this subset of the PI 3-kinase family are able to phosphorylate lipids. However, all members of this family have been shown to possess serine/threonine kinase activity. ATM phosphorylates key proteins involved in a variety of cell-cycle checkpoint signalling pathways that are initiated in response to DNA DSBs production (see below). These downstream effector proteins include p53, Chk2, NBS1/nibrin, BRCA1 and Rad 17 (Abraham, 2001)

ATM is the product of the gene mutated in ataxia-telangiectasia (A-T) (Savitsky, et al. (1995)). A-T is a human autosomal recessive disorder present at an incidence of around 1 in 100,000 in the population. A-T is characterised by a number of debilitating symptoms, including progressive cerebellar degeneration, occulocutaneous telangiectasia, growth retardation, immune deficiencies, cancer predisposition and certain characteristics of premature ageing (Lavin and Shiloh, Annu. Rev. Immunol., 15,177-202 (1997); Shiloh, Curr. Opin. Genet. Dev., 11, 71-77 (2001)). At the cellular level, A-T is characterised by a high degree of chromosomal instability, radio-resistant DNA synthesis, and hypersensitivity to ionizing radiation (IR) and radiomimetic drugs. In addition, A-T cells are defective in the radiation induced G₁-S, S, and G₂-M cell cycle checkpoints that are thought to arrest the cell cycle in response to DNA damage in order to allow repair of the genome prior to DNA replication or mitosis (Lavin and Shiloh, 1997). This may in part reflect the fact that A-T cells exhibit deficient or severely delayed induction of p53 in response to IR. Indeed, p53-mediated downstream events are also defective in A-T cells following IR exposure. ATM therefore acts upstream of p53 in an IR-induced DNA damage signalling pathway. A-T cells have also been shown to accumulate DNA double-strand breaks (DSBs) after ionizing radiation, suggesting a defect in DSB repair.

It is clear that ATM is a key regulator of the cellular response to DNA DSBs. Therefore the inhibition of this kinase through small molecules will sensitise cells to both ionising radiation and to chemotherapeutics that induce DNA DSBs either directly or indirectly. ATM inhibitors may thus be used as adjuncts in cancer radiotherapy and chemotherapy. To date the only reported inhibitors of ATM (caffeine and wortmannin; Sarkaria, et al., Cancer Res., 59, 4375-4382 (1999); Banin, et al., Science, 281,1674-1677 (1998)) do cause radiosensitisation but it is unclear whether this mechanism of action is mediated through ATM inhibition as these small molecules are very non-specific in action as kinase inhibitors.

ATM function in response to ionising radiation induced DNA damage has been shown to be tissue specific. For example, while fibroblasts derived from Atm null mice are radiosensitive

Atm null neurons are radioresistant through a lack of IR induced apoptosis (Herzog, et al., Science, 280, 1089-1091 (1998)). Therefore, inhibitors of ATM have the potential to be radio-protective in specific cellular contexts.

ATM inhibitors may also prove useful in the treatment of retroviral mediated diseases. It has been demonstrated that a deficiency in ATM protein sensitises cells to retrovirus induced cell death and that a small molecule inhibitor of ATM kinase function is capable of suppressing the replication of both wild-type and drug-resistant HIV-1 (Lau, et al., Nature Cell Biology, 7, 493-500 (2005). Therefore ATM inhibitors have the potential to block retroviral DNA integration.

ATM is known to play a crucial role in controlling the length of telomeric chromosomal ends (Metcalfe, et al., Nat Genet., 13, 350-353 (1996)). Telomeric ends in most normal cell types shorten at each cell division. Cells with excessively shortened telomeres are unable to divide. Inhibitors of ATM may therefore, have utility in preventing cancer progression by limiting the growth potential of cancerous or pre-cancerous cells. Furthermore, ATM does not appear to be part of the telomerase enzyme itself (Metcalfe, et al. (1996)) Therefore it is likely that ATM inhibitors will work synergistically with anti-telomerase drugs.

Cells derived from A-T patients or from mice null for ATM grow slower in culture than genetically matched ATM positive cells. Therefore an ATM inhibitor may have growth inhibitory/anti-proliferative properties in its own right. Therefore an ATM inhibitor may be used as a cytostatic agent in the treatment of cancer.

A-T patients display immuno-deficiencies, demonstrating that ATM is required for generation of a fully functional immune system. Inhibitors of ATM may, therefore, be used in modulating the immune system.

In summary ATM inhibitors have the potential to sensitise tumour cells to ionising radiation or DNA DSB inducing chemotherapeutics, to modulate telomere length control mechanisms, to block retroviral integration, modulate the immune system and to protect certain cell types from DNA damage induced apoptosis.

Some of the present inventors have previously described a broad class of compounds which exhibit inhibition of ATM. These are described in a published PCT application, WO 03/070726 (incorporated herein by reference). The ATM inhibitors have the following general structure:

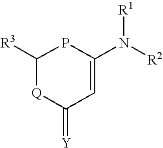

Further ATM inhibitors from within that broad class of compounds are described in the published PCT application, WO 2005/016919 (incorporated herein by reference). ATM inhibitors described in WO 03/070726 and WO 2005/016919 were shown to sensitise cells to ionising radiation or DNA double strand break chemotherapies and to inhibit retroviral transduction and replication.

A key problem facing any drug-based treatment is the efficient delivery of the drug from its site of administration to its intended site of action. Intravenous administration permits direct access of the drug to the plasma. However, in many situations it is preferable to administer a drug orally, so as to minimise patient discomfort. Absorption of a drug from gut to plasma is influenced by a wide variety of factors, including the physicochemical properties of the drug and its formulation. It is desirable that a compound for use in a drug-based method of treatment has acceptable oral bioavailability.

In the treatment of solid tumours it is desirable that a drug reaches an effective concentration in the tumour tissue following administration of the drug to a patient. This partitioning of drug to a tumour depends, amongst other things, on the physicochemical properties of the drug, its plasma half-life and other pharmacokinetic factors such as the rate of clearance of the drug from the body. It is desirable that a compound for use in a drug-based method of treatment of a tumour exhibits acceptable pharmacokinetic properties.

In any drug-based treatment it is desirable that the drug's toxicity to the organism as a whole be minimised. This is especially important in the case of agents used in the treatment of cancer or viral infection. It is preferable that a compound for use in a drug-based method of treatment exhibits an acceptable maximum tolerable dose.

The present inventors have now surprisingly discovered that a compound failing within the broad class of ATM inhibitor compounds described previously, but not specifically exemplified, is superior to other compounds from that class in respect of at least one property that is desirable in a compound for use in a drug-based treatment (e.g. oral bioavailability, tissue pharmacokinetics, maximum tolerable dose). Accordingly, the first aspect of the invention provides a compound of formula (I): 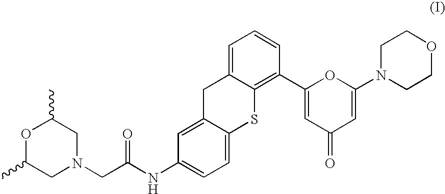
and isomers, salts, solvates, chemically protected forms and prodrugs thereof. The compound of formula (I) has the chemical name: 2-(2,6-Dimethyl-morpholin-4-yl)-N-[5-(6-morpholin-4-yl-4-oxo-4H-pyran-2-yl)-9H-thioxanthen-2-yl]-acetamide.

In particular, the following isomers of the compound of formula (I) are of interest:

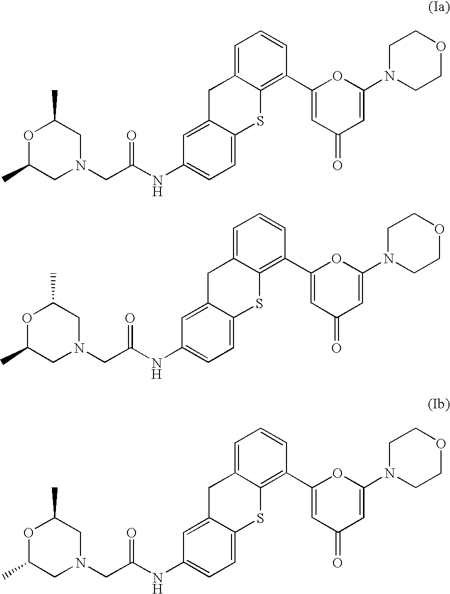

The compound of formula (Ia) is the cis-form, 2-((2R,6S)-2,6-dimethyl-morpholin-4-yl)-N-[5-(6-morpholin-4-yl-4-oxo-4H-pyran-2-yl)-9H-thioxanthen-2-yl]-acetamide. The two diastereoisomers of the compound of formula (Ib) are the trans-form, 2-((2S,6S)-2,6-dimethyl-morpholin-4-yl)-N-[5-(6-morpholin-4-yl-4-oxo-4H-pyran-2-yl)-9H-thioxanthen-2-yl]-acetamide and 2-((2R,6R)-2,6-dimethyl-morpholin-4-yl)-N-[5-(6-morpholin-4-yl-4-oxo-4H-pyran-2-yl)-9H-thioxanthen-2-yl]-acetamide.

Particularly preferred is the isomeric form corresponding to 2-((2R,6S)-2,6-Dimethyl-morpholin-4-yl)-N-[5-(6-morpholin-4-yl-4-oxo-4H-pyran-2-yl)-9H-thioxanthen-2-yl]-acetamide (Ia).

A second aspect of the invention provides a composition comprising the compound of the first aspect of the invention and a pharmaceutically acceptable carrier or diluent.

A third aspect of the invention provides the use of the compound of the first aspect of the invention or a composition of the second aspect of the invention in a method of therapy.

A fourth aspect of the invention provides the use of the compound of the first aspect of the invention or a composition of the second aspect of the invention in the preparation of a medicament for treating diseases which are ameliorated by the inhibition of ATM.

A fifth aspect of the invention provides for the use of the compound of the first aspect of the invention or a composition of the second aspect of the invention in the preparation of a medicament for use as an adjunct in cancer therapy or for potentiating tumour cells for treatment with ionising radiation or chemotherapeutic agents. In some embodiments, the type of cells senstized are mismatch repair (MMR) negative tumour cells, and in particular MMR negative colorectal carcinoma cells.

The fifth aspect of the invention also provides the compound of the first aspect of the invention or a composition of the second aspect of the invention in combination with ionising radiation or chemotherapeutic agents for use in the treatment of cancer.

A sixth aspect of the invention provides for the use of the compound of the first aspect of the invention or a composition of the second aspect of the invention in the preparation of a medicament for the treatment of retroviral mediated diseases or disease ameliorated by the inhibition of ATM, which include: acquired immunodeficiency syndrome and hyperproliferative disease and conditions, as described below.

The sixth aspect of the invention also provides the compound of the first aspect of the invention or a composition of the second aspect of the invention for use in the treatment of retroviral mediated diseases or diseases ameliorated by the inhibition of ATM, which include: acquired immunodeficiency syndrome and hyperproliferative disease and conditions, as described below.

A seventh aspect of the invention provides the active compound as described herein for use in a method of treatment of the human or animal body, preferably in the form of a pharmaceutical composition.

An eighth aspect of the invention provides a method of inhibiting ATM in vitro or in vivo, comprising contacting a cell with an effective amount of the active compound as described herein.

A ninth aspect of the invention provides a method of synthesising compound (I) as defined in claim 1, which method comprises reacting 2-(7-amino-9H-thioxanthen-4-yl)-6-morpholin-4-yl-pyran-4-one with chloracetyl chloride followed by 2,6-dimethylmorpholine. If the compound to be synthesised is of formula (Ia) then cis 2,6-dimethylmorpholine is used. If the compound to be synthesised is of formula (Ib) then trans 2,6-dimethylmorpholine is used.

### BRIEF DESCRIPTION OF THE DRAWINGS

FIG. 1 is a graph showing the percentage of cellular survival in response to the topoisomerase I inhibitor camptothecin.

### DEFINITIONS

Isomers, Salts, Solvates, Protected Forms, and Prodrugs

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R—, S—, and meso-forms; D- and L-forms; d- and I-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

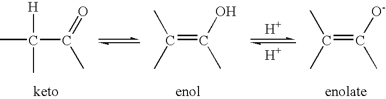

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g., fractional crystallisation and chromatographic means) of such isomeric forms are either known in the art or are readily obtained by adapting the methods taught herein, or known methods, in a known manner.

The compound of the first aspect of the invention includes isomers of formula (I) as described above. For example, 2-((2S,6S)-2,6-Dimethyl-morpholin-4-yl)-N-[5-(6-morpholin-4-yl-4-oxo-4H-pyran-2-yl)-9H-thioxanthen-2-yl]-acetamide and 2-((2R,6R)-2,6-Dimethyl-morpholin-4-yl)-N-[5-(6-morpholin-4-yl-4-oxo-4H-pyran-2-yl)-9H-thioxanthen-2-yl]-acetamide. Particularly preferred is the isomeric form corresponding to 2-((2R,6S)-2,6-Dimethyl-morpholin-4-yl)-N-[5-(6-morpholin-4-yl-4-oxo-4H-pyran-2-yl)-9H-thioxanthen-2-yl]-acetamide.

Unless otherwise specified, a reference to a particular compound also includes ionic, salt, solvate, and protected forms of thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci., 66, 1-19 (1977).

For example, a functional group of the compound which may be anionic can be used to form a salt with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Similarly, a functional group of the compound which may be cationic may be used to form a salt with a suitable anion. Examples of suitable inorganic anions include, but are not limited to, those derived from the following inorganic acids: hydrochloric, hydrobromic, hydroiodic, sulphuric, sulphurous, nitric, nitrous, phosphoric, and phosphorous. Examples of suitable organic anions include, but are not limited to, those derived from the following organic acids: acetic, propionic, succinic, glycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, phenylsulfonic, toluenesulfonic, methanesulfonic, ethanesulfonic, ethane disulfonic, oxalic, pantothenic, isethionic, valeric, lactobionic, and gluconic. Examples of suitable polymeric anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form”, as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts, Wiley, 1999).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

It may be convenient or desirable to prepare, purify, and/or handle the active compound in the form of a prodrug. The term “prodrug”, as used herein, pertains to a compound which, when metabolised (e.g. in vivo), yields the desired active compound. Typically, the prodrug is inactive, or less active than the active compound, but may provide advantageous handling, administration, or metabolic properties.

For example, some prodrugs are esters of the active compound (e.g. a physiologically acceptable metabolically labile ester). During metabolism, the ester group is cleaved to yield the active drug.

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

Acronyms

For convenience, many chemical moieties are represented using well known abbreviations, including but not limited to, methyl (Me), ethyl (Et), n-propyl (nPr), iso-propyl (iPr), n-butyl (nBu), tert-butyl (tBu), n-hexyl (nHex), cyclohexyl (cHex), phenyl (Ph), biphenyl (biPh), benzyl (Bn), naphthyl (naph), methoxy (MeO), ethoxy (EtO), benzoyl (Bz), acetyl (Ac), 1,3-bis(diphenylphosphino) propane (dppf).

For convenience, many chemical compounds are represented using well known abbreviations, including but not limited to, methanol (MeOH), ethanol (EtOH), iso-propanol (i-PrOH), methyl ethyl ketone (MEK), ether or diethyl ether (Et₂O), acetic acid (AcOH), dichloromethane (methylene chloride, DCM), trifluoroacetic acid (TFA), dimethylformamide (DMF), tetrahydrofuran (THF), and dimethylsulfoxide (DMSO).

Use of the Compound of the Invention

The term “active”, as used herein, pertains to a compound of the first aspect of the invention which is capable of inhibiting ATM activity, and specifically includes both compound (I) with its intrinsic activity (drug) as well as prodrugs of compound (I), which prodrugs may themselves exhibit little or no intrinsic activity.

One assay which may be used in order to assess the ATM inhibition offered by a particular compound is described in example 3 below.

The present invention provides a method of inhibiting ATM in a cell, comprising contacting said cell with an effective amount of the active compound, preferably in the form of a pharmaceutically acceptable composition. Such a method may be practised in vitro or in vivo.

For example, a sample of cells (e.g. from a tumour) may be grown in vitro and the active compound brought into contact with said cells in conjunction with agents that have a known curative effect, and the enhancement of the curative effect of the compound on those cells observed.

The invention further provides the active compound for use in a method of treatment of the human or animal body. Such a method may comprise administering to such a subject a therapeutically-effective amount of the compound, preferably in the form of a pharmaceutical composition.

The term “treatment” as used herein in the context of treating a condition, pertains generally to treatment and therapy, whether of a human or an animal (e.g. in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e. prophylaxis) is also included.

The term “therapeutically-effective amount” as used herein, pertains to that amount of the active compound, or a material, composition or dosage form comprising the active compound, which is effective for producing some desired therapeutic effect, commensurate with a reasonable benefit/risk ratio.

The term “adjunct” as used herein relates to the use of the active compound in conjunction with known therapeutic means. Such means include cytotoxic regimens of drugs and/or ionising radiation as used in the treatment of different cancer types. In particular, the active compound will potentiate the actions of a number of cancer chemotherapy treatments, which include, but are not limited to, the topoisomerase class of poisons and any chemotherapeutic that will induce a DNA double strand break used in treating cancer. Topoisomerase I inhibitors that may be used in combination with the active compound include the camptothecin compounds, e.g. topotecan (Hycamtin), irinotecan (CPT11—Camptosar), rubitecan and exatecan. Dual Topoisomerase I and II inhibitors that may be used in combination with the active compound include benzophenainse, XR 11576/MLN 576 and benzopyridoindoles. Topoisomerase II inhibitors that may be used in combination with the active compound include the intercalators and DNA binders Doxorubicin, Danorubicin, and other rubicins, the acridines (Amsacrine, m-AMSA), plus Mitoxantrone and AQ4. Non-intercalators which are topoisomerase II inhibitors include Etopside and Teniposide (epipodophyllotoxins).

Other, but less preferred, possible chemotherapeutic agents with which the compound of formula (I) may be combined include one or more of the following categories of anti-tumour agents:

(i) other antiproliferative/antineoplastic drugs and combinations thereof, as used in medical oncology, such as alkylating agents (for example cis platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5 fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5*-reductase such as finasteride;

(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);

(iv) inhibitors of growth factor function: for example such inhibitors include growth factor antibodies and growth factor receptor antibodies (for example the anti erbB2 antibody trastuzumab [Herceptin™], the anti-EGFR antibody panitumumab, the anti erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI 774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;

(v) antiangiogenic agents such as those which inhibit the effects of vascular endothelial growth factor, [for example the anti vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856 and WO 98/13354 and compounds that work by other mechanisms (for example linomide, inhibitors of integrin avb3 function and angiostatin)];

(vi) vascular damaging agents such as Combretastatin A4 and compounds disclosed in International Patent Applications WO 99/02166, WO 00/40529, WO 00/41669, WO 01/92224, WO 02/04434 and WO 02/08213;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene directed enzyme pro drug therapy) approaches such as those using cytosine deaminase, thymidine kinase or a bacterial nitroreductase enzyme and approaches to increase patient tolerance to chemotherapy or radiotherapy such as multi drug resistance gene therapy; and

(ix) immunotherapy approaches, including for example ex vivo and in vivo approaches to increase the immunogenicity of patient tumour cells, such as transfection with cytokines such as interleukin 2, interleukin 4 or granulocyte macrophage colony stimulating factor, approaches to decrease T cell anergy, approaches using transfected immune cells such as cytokine transfected dendritic cells, approaches using cytokine transfected tumour cell lines and approaches using anti idiotypic antibodies.

The compound of formula (I) can be used in the treatment of proliferative and hyperproliferative diseases/conditions, either alone or in combination, as described above, examples of which include the following cancers:

(1) carcinoma, including that of the bladder, brain, breast, colon, kidney, liver, lung, ovary, pancreas, prostate, stomach, cervix, colon, thyroid and skin;

(2) hematopoietic tumors of lymphoid lineage, including acute lymphocytic leukaemia, B-cell lymphoma and Burketts lymphoma;

(3) hematopoietic tumours of myeloid lineage, including acute and chronic myelogenous leukaemias and promyelocytic leukaemia;

(4) tumours of mesenchymal origin, including fibrosarcoma and rhabdomyosarcoma; and

(5) other tumours, including melanoma, seminoma, tetratocarcinoma, neuroblastoma and glioma.

The present inventors have found that the compound of the present invention preferentially sensitized mismatch repair (MMR) negative cells to the topoisomerase I inhibitor camptothecin compared to a matched MMR positive cell line as judged by a clonogenic survival assay. This is described in detail in example 9. Therefore, it is preferred that the compound of formula (I) is used in the treatment of MMR negative tumour cells, and more particularly MMR negative colorectal tumour cells.

The present inventors have previously found that ATM inhibitory compounds of similar structure to that of the present invention can efficiently repress retroviral vector transduction in one-step, cell based integration assays (termed LUCIA) and inhibit HIV-1 infection in 4-day replication assays at sub-micromolar concentrations (Lau, et al., Nature Cell Biology, 7, 493-500 (2005). Further, in contrast to the observations of Daniel et al., where it was concluded that the effect of ATM on retroviral integration would only be seen in a DNA-PK-deficient background, this effect works in the presence of functional DNA-PK activity. The compound of the present invention has also been shown to repress retroviral transduction at sub-micromolar concentrations, as described in example 4 below.

Initial linkage of linear retroviral DNA with host cell chromosomal DNA is catalysed by viral integrase (IN) and results in short staggered DNA strand breaks in the host cell DNA at the site of attachment (Brown, P. O. (1990) Integration of retroviral DNA. Curr Top Microbiol Immunol, 157, 19-48). These gapped DNA intermediates are shown to be sensed as sites of DNA damage by the host cell and repaired by the ATM pathway to complete the process of integration and allow productive infection to occur. The active compound would be able to prevent the repair of gapped DNA intermediates by the ATM pathway and thus prevent complete integration of retroviral DNA into the host genome.

As described above, the invention provides a compound for use in the treatment of retroviral infection and the use of such a compound in the manufacture of a medicament for use in the treatment of retroviral infection.

Also provided by the invention is a method of treatment of a retroviral infection comprising administering the active compound, preferably in the form of a pharmaceutical composition, to an individual in need thereof.

Retroviral mediated diseases which may be treated as described above include HIV infection and acquired immunodeficiency syndrome (AIDS) and Human T-cell Leukaemia virus (HTLV) infection and its associated diseases adult T-cell leukaemia/lymphoma (ATLL) and tropical spastic paraparesis/HTLV-1 associated myelopathy (TSP/HAM).

The active compound may be used in combination with other retroviral therapies to suppress virus replication, for example in a ‘highly active anti-retroviral therapy’ or HAART treatment.

The invention provides a pharmaceutical composition comprising the active compound as described herein and one or more other anti-retroviral agents.

The invention also provides a composition comprising the active compound and one or more other anti-retroviral agents for treatment of a retroviral infection and the use of such a composition in the manufacture of a medicament for use in the treatment of a retroviral infection.

Suitable anti-retroviral agents which inhibit retroviral replication, for example retroviral protease inhibitors (PI) such as Sequinavir, Indinavir, Ritonavir and Nelfinavir, nucleoside retroviral reverse transcriptase inhibitors such as 3′-azido-3′deoxythymidine (AZT; Zidovudine), 2′,3′-Dideoxycytosine (ddC; Zalcitabine), 2′,3′-Dideoxyinosine (ddI; Didanosine)and 3TC; (Lamivudine), and non-nucleoside retroviral reverse transcriptase inhibitors such as Nevirapine, Delavirdine and Efavirenz.

Administration

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly. Preferably, the active compound is administered orally or intravenously.

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human. Preferably, the subject is human.

Formulations

While it is possible for the active compound to be administered alone, it is preferable to present it as a pharmaceutical composition (e.g. formulation) comprising the active compound together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing the active compound, together with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, losenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g. by ingestion) may be presented as discrete units such as capsules, cachets or tablets, each containing a predetermined amount of the compound of the invention; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or moulding, optionally with one or more accessory ingredients. Compressed tablets may be prepared by compressing in a suitable machine the compound of the invention in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Moulded tablets may be made by moulding in a suitable machine a mixture of the powdered compound moistened with an inert liquid diluent. The tablets may optionally be coated or scored and may be formulated so as to provide slow or controlled release of the compound of the invention therein using, for example, hydroxypropylmethyl cellulose in varying proportions to provide the desired release profile. Tablets may optionally be provided with an enteric coating, to provide release in parts of the gut other than the stomach.

Preferred solid formulations are shown in the table below, in which the percentage of each component is given for four different formulations. The formulations numbered 3 and 4 are particularly preferred.

| Component | Use | 1 | 2 | 3 | 4 |
|---|---|---|---|---|---|
| Compound (Ia) | Active | 43.0 | 43.0 | 43.0 | 43.0 |
| Lactose | Diluent | 51.0 | 50.0 | 49.75 | 45.0 |
| Sodium starch | Disintegrant | 5.0 | 5.0 | 5.0 | 5.0 |
| glycollate | | | | | |
| Magnesium stearate | Lubricant | 1.0 | 2.0 | 2.0 | 2.0 |
| Sodium lauryl sulphate | Wetting agent | — | — | 0.25 | — |
| Microcrystalline | Disintegrant/ | — | — | — | 5.0 |
| cellulose | lubricant | | | | |

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with the compound of the invention and optionally one or more excipients or diluents.

Formulations suitable for topical administration in the mouth include losenges comprising the active compound in a flavoured basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required.

Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g. by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Dosage

It will be appreciated that appropriate dosages of the active compound, and compositions comprising the active compound, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Preferably, the dose of the active compound is in the range of about 100 μg to about 50 mg per kilogram body weight of the subject per day. Where the active compound is a salt, an ester, prodrug, or the like, the amount administered is calculated on the basis of the parent compound and so the actual weight to be used is increased proportionately.

### EXAMPLES

The following examples are provided solely to illustrate the present invention and are not intended to limit the scope of the invention, as described herein.

### Example 1

### Synthesis of 2-((2R,6S)-2,6-Dimethyl-morpholin-4-yl)-N-[5-(6-morpholin-4-yl-4-oxo-4H-pyran-2-yl)-9H-thioxanthen-2-yl]-acetamide (compound (Ia))

Compound (Ia) was synthesised by reacting 2-(7-Amino-9H-thioxanthen-4-yl)-6-morpholin-4-yl-pyran-4-one with chloracetyl chloride followed by cis 2,6-dimethylmorpholine as shown in the scheme below:

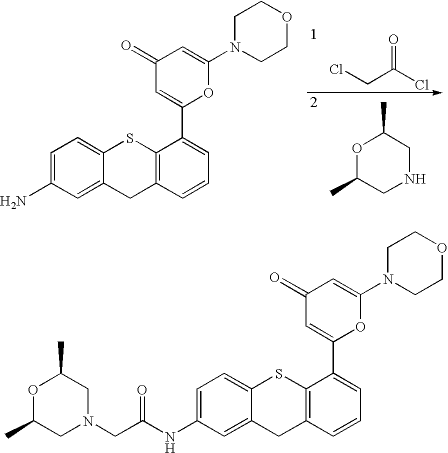

The synthesis of 2-(7-Amino-9H-thioxanthen-4-yl)-6-morpholin-4-yl-pyran-4-one was performed as described in WO 03/070726 (see Example 9, compound 20 therein), which is incorporated herein by reference.

To a solution of 2-(7-Amino-9H-thioxanthen-4-yl)-6-morpholin-4-yl-pyran-4-one (20 mg, 0.051 mmol) in dry DMA (0.55 ml) was added chloroacetyl chloride (4.49 μl, 0.056 mmol) and triethylamine (15.7 μl, 0.11 mmol). The mixture was stirred at room temperature for 90 minutes. To the reaction was then added the Cis 2,6-dimethylmorpholine and the mixture stirred at room temperature overnight.

The crude mixture was then submitted for HPLC purification and mass spectrometer analysis as described below.

The product was purified on Gilson LC units.

Mobile phase A—0.1% aqueous TFA, Mobile phase B—Acetonitrile, Flow rate 6 ml/min., Gradient—typically starting at 90% A/10% B for one minute, rising to 97% B after 15 minutes, holding there for 2 minutes, then back to the starting conditions. Column: Jones Chromatography Genesis 4μ C18 column, 10 mm×250 mm. Peak acquisition based on UV detection at 254 nm.

Mass Specs were recorded on a Finnegan LCQ instrument in positive ion mode. Mobile phase A—0.1% aqueous formic acid, Mobile phase B—Acetonitrile, Flow rate 2 ml/min., Gradient—starting at 95% A/5% B for one minute, rising to 98% B after 5 minutes, holding there for 3 minutes, then back to the starting conditions. Column—Phenomenex 5μ Luna C18 column, 4.6 mm×50 mm UV detection at 254 nm, PDA detection scanning from 210 to 600 nm.

The results of the analysis were as follows:

Purity=85%, retention time (mins)=3.33, M⁺+1=548

### Example 2

### Scaled-up synthesis of 2-((2R,6S)-2,6-Dimethyl-morpholin-4-yl)-N-[5-(6-morpholin-4-yl-4-oxo-4H-pyran-2-yl)-9H-thioxanthen-2-yl]-acetamide (compound (Ia))

Compound (Ia) was synthesised according to the scheme shown below:

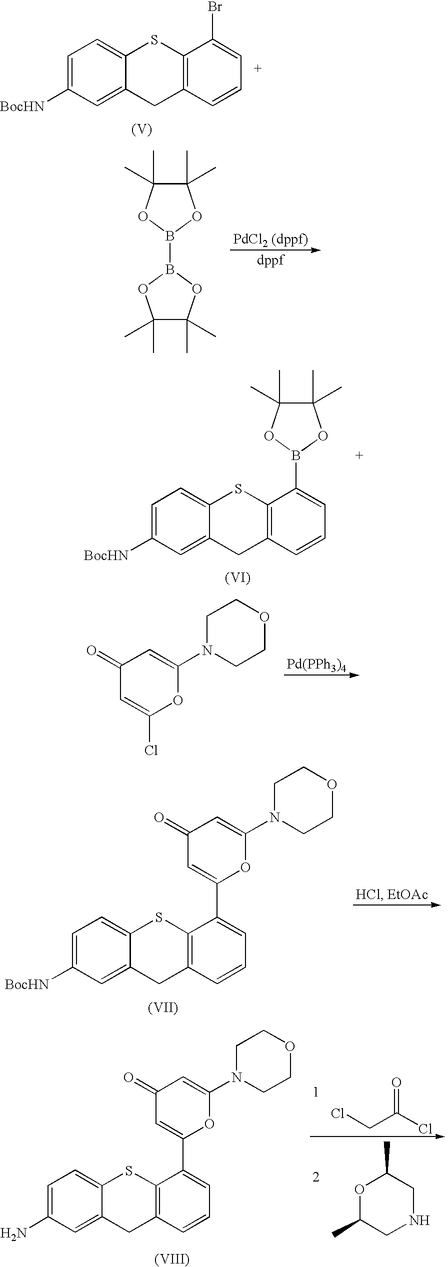

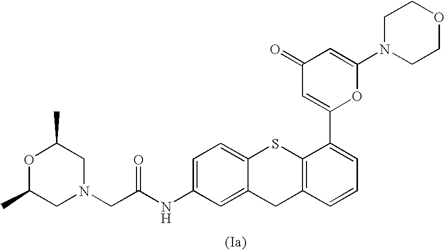

At each stage of the synthesis, reaction products were analysed by HPLC. The following HPLC conditions were employed:

| System | Agilent1100 series liquid chromato- | | |
|---|---|---|---|
| | graph, or equivalent | | |
| Column | Synergi Fusion-RP 80A 150 mm × 4.6 | | |
| | mm 4 μm particle | | |
| Mobile phase A | Purified Water:acetonitrile:formic | | |
| | acid (95:5:0.1) | | |
| Mobile phase B | Purified Water:acetonitrile:formic | | |
| | acid (5:95:0.1) | | |
| Flow rate | 1.0 ml · min⁻¹ | | |
| Injection volume | 5 μl | | |
| Detection | UV at 254 nm | | |
| Column temperature: | 30° C. | | |
| Post run | 5 minutes | | |
| Gradient | Time | % A | % B |
| | 0 | 100 | 0 |
| | 5 | 100 | 0 |
| | 15 | 0 | 100 |
| | 25 | 0 | 100 |
| | 27 | 100 | 0 |

| | |
|---|---|
| Diluent: | Acetonitrile |

Sample Preparation:

Approximately 50 mg of sample was weighed into a 50 ml volumetric flask. This was then dissolved in minimum amount of dimethylformamide and make up to volume with diluent mixed well.

Typical Retention Times:
(V) 16.9 minutes
(VI) 17.1 minutes
(VII) 16.1 minutes
(VII) 12.4 minutes
(Ia) 11.6 minutes

Synthesis of (V)

The synthesis of (V) is described in WO 2005/016919 (see Example 5 therein), which is incorporate herein by reference.

Synthesis of (VI)

To two 5 L flasks was charged bis(pinacolato)diboron (172.5 g), dichlorobis(diphenyl phosphine) ferrocene palladium (II) (26.0 g), bis(diphenylphosphine) ferrocene (18.0 g) and potassium acetate (187.5 g). DMF (2 L, degassed with N₂ and sonicated for 30 mins) was added, followed by stage 5 (250 g). The reaction was then heated overnight at 100° C. HPLC analysis showed both the reactions to be complete.

The reactions were cooled to 25° C. and both poured into the same water (10 L) [exothermed to 28° C.]. After 30 mins stirring the solids were filtered off. The solids were washed with water (10 L) and then dissolved in DCM (6.25 L). The organic layer was washed with water (3×6.25 L), dried over MgSO₄ (143 g), filtered and concentrated in vacuo to give 807 g purple solid (HPLC: Stage 5: 0.88%, Stage 6: 59.93%).

The solid was redissolved in DCM (1.8 L) and then split into 2 equal batches. Each batch was columned on silica (1.5 kg) using DCM as the eluent. The product was collected in fractions 6-26 (1 L fractions). The solvent was removed to give 413.9 g tan solid (HPLC: 70.24%).

Synthesis of (VII)

A 5 L reaction was set up and purged with nitrogen. (VI) (413.9 g), 1,4-dioxane (3306 ml), 2-chloropyranone (223.4 g) and K₂CO₃ (388.4 g) were charged. The slurry was sparged with nitrogen for 30 mins before the addition of Pd(PPh₃)₄ (54.3 g). The reaction was then heated at 96° C. overnight. (HPLC: 8.3% chloropyranone, 62.0% (VII) and 12.4% (VI)).

A further 20 g 2-chloropyranone was added and the reaction heated for a further 5 hours. (HPLC: 5.1% chloropyranone, 74.5% (VII) and 4.5% (VI)). The reaction was allowed to cool slowly to room temperature overnight. The reaction was filtered and the filter cake washed with DCM (3×1.3 L). The filtrate was stripped to dryness to give a dark sticky solid. This was redissolved in DCM (7 L) and then the solvent removed to give a total of 640.4 g dark crystalline solid. This was split into 3 roughly equal batches and each purified by column chromatography on silica (2.5 kg). The column was set up in EtOAc (10 L) and the crude (VII) charged to the column as a solution in EtOAc (700 ml). The product was eluted using 8% methanol in ethyl acetate (32 L). The fractions containing product (16-30) were combined and concentrated on a rotary evaporator.

| Column No. | Crude (VII) charged | Yield | Comments |
|---|---|---|---|
| 1 | 229.9 g | 107.3 g | 93.1% by HPLC |
| 2 | 208.2 g | 88.3 g | 93.4% by HPLC |
| 3 | 202.3 g | 95.8 g | 94.9% by HPLC |

Synthesis of (VIII)

To a solution of 4 M HCl in EtOAc (4 L) was added (VII) (282.2 g) over 20 mins and the reaction stirred overnight at room temperature. (HPLC: 90.28% Stage 8, Stage 7 not detected, 7.5% impurity). The reaction was filtered and washed with EtOAc (2.7 L). The solid was partitioned between DCM (3.3 L) and water (3.3 L). The pH was then adjusted to 9-10 with the addition of solid K₂CO₃ (235 g). The DCM layer was removed and the aqueous layer extracted with DCM (2×3.3 L). The DCM layer was dried over MgSO4 (80 g), filtered and concentrated in vacuo to give 271 g yellow solid. This was dissolved in DCM (2.5 L) and adsorbed onto silica (550 g).

The solid was purified by column chromatography on silica (6 kg) using as eluent: 4% MeOH/EtOAc (130 L), 10% MeOH/EtOAc (10 L), 20% MeOH/EtOAc (20 L) and 40% MeOH/EtOAc (40 L). The product fractions (150-195 L) were concentrated to give 198 g solid. This was dried in a vacuum oven at 50° C. until constant weight, giving 178 g yellow solid. (98.9% by HPLC, 79.2% yield).

### Synthesis of (Ia): 2-((2R,6S)-2,6-Dimethyl-morpholin-4-yl)-N-[5-(6-morpholin-4-yl-4-oxo-4H-pyran-2-yl)-9H-thioxanthen-2-yl]-acetamide (Compound (Ia))

In a 5 L flask under N₂ was charged (VIII) (173.4 g) and chloroform (1673 ml). To the solution was added Na₂CO₃ (140.6 g) and the reaction stirred for 10 mins. A solution of chloroacetyl chloride (35.2 ml) in CHCl₃ (169 ml) was added over 15 mins at <20° C. The reaction was stirred for 1.5 hours at room temperature (HPLC: 1.1% Stage 8, 96.8% Intermediate). Cis 2,6-dimethylmorpholine (108.4 ml) was added over 20 mins at <20° C. and then the reaction heated at 55-60° C. overnight. (HPLC: 91.1% Stage 9, 1.1% Stage 8, 4.2% Intermediate). Cis 2,6-dimethylmorpholine (6 ml) was added and the reaction heated for a further 1 hour (HPLC: 95.2% Stage 9, 1.0% Stage 8, 0.5% Intermediate). The reaction was allowed to cool to room temperature and water (1673 ml) added. After stirring for 20 mins, the organic layer was separated and then washed with water (2×1 L). The organic layer was dried over MgSO₄ (50 g), filtered and the solvent removed in vacuo to give a brown oil. This was dissolved in DCM (1.1 L) and TBME (2.2 L) added to give a slurry. The solvents were then removed in vacuo to give a tan solid (441 g). This was slurried in TBME (2.8 L) at 35° C. for 30 mins, before cooling to 10° C. and filtering. The filter cake was washed with TBME (2×450 ml) and pulled dry to give 308 g solid. This was dried at 45° C. in a vacuum oven for 2 days until constant weight. This gave 247 g off-white solid which was subjected to analysis by ¹H NMR, mass spec. and HPLC.

¹H-NMR spectra were recorded on a Bruker AC3000 Series NMR 300 MHz spectrometer instrument. Chemical shifts were referenced relative to tetramethylsilane in CDCl₃. The product was found to contain ˜12% TBME by ¹H NMR. Excluding the TBME, the purity of the final product was >95% by ¹H NMR. The ¹H NMR spectrum of the product conformed to the structure of compound (Ia). The following chemical shift data were obtained:

9.10 (s, 1H), 7.84 (s, 1H), 7.33 (m, 6H), 6.32 (s, 1H), 5.51 (s, 1H), 3.90 (s, 2H), 3.82 (m, 4H), 3.73 (m, 2H), 3.20 (s, 2H), 3.11 (s, 2H), 2.73 (d, J=10.4 Hz, 2H), 2.03 (t, J=10.4 Hz, 2H), 1.16 (s, 6H)

The mass spectrum conformed to the structure of formula (Ia).

The purity of the final product was 95.8% by HPLC

### Example 3

### Synthesis of 2-((2R,6R)-2,6-Dimethyl-morpholin-4-yl)-N-[5-(6-morpholin-4-yl-4-oxo-4H-pyran-2-yl)-9H-thioxanthen-2-yl]-acetamide (Compound (Ib)

A batch of compound (I) was synthesised from an approximately 70:30 mixture of cis- and trans-dimethylmorpholine isomers. This material was subjected to additional purification in order to isolate the trans-isomer of compound (I), i.e. compound (Ib).

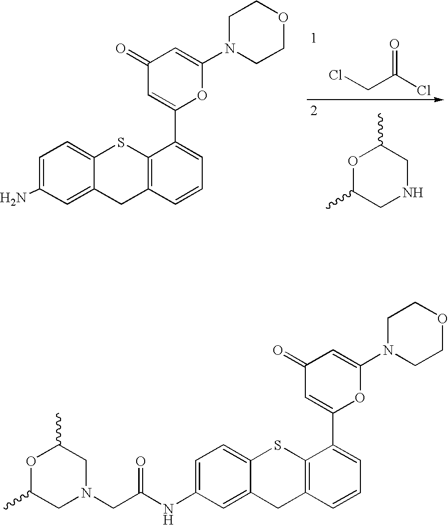

To a solution of 2-(7-amino-9H-thioxanthen-4-yl)-6-morpholin-4-yl-pyran-4-one (200 mg, 1 eq.) in CHCl₃ (6 mL) in a large reactivial was added sodium carbonate (162 mg, 3 eq.) in one portion, followed by the addition of chloroacetyl chloride (43 μL, 1.05 eq.) dropwise over 1 minute. After 1 hour, 2,6-dimethylmorpholine [Aldrich isomeric batch, 70:30 mixture of cis- and trans-dimethylmorpholine isomers] (75 μL, 1.2 eq.) was added in one portion, and the reactivial was heated in an oil bath at 70° C., and the reaction was allowed to stir at reflux overnight.

The reaction was allowed to cool to room temperature, quenched by the addition of sat. aq. NaHCO₃ (˜10 mL), the organic layer was then removed and the aqueous layer was then extracted with DCM (3×10 mL). The combined organics were washed with saturated brine (1×15 mL), dried over MgSO₄, filtered and concentrated on a buchi, leaving a mid-brown oil. This was subjected to flash chromatography [1% MeOH/DCM, then 2%, 5%, 10%], leaving a light yellow crystalline solid, which was dried in the drying piston. LC-MS showed that the desired product ran at 3.45 mins rt with a m/z of 548 [ES+].

The LC conditions used were:

Mobile phase A: 0.1% Formic acid in water

Mobile phase B: 0.1% Formic acid in acetonitrile

Gradient: 10% B for 4 minutes, then to 95% B over 15.5 minutes, hold for 3.5 minutes. Flow rate 6 ml/min.

Column: Genesis C18 250 mm×10 mm, 4 um.

### Example 4

### In Vitro ATM Inhibition Assay

In order to assess the inhibitory action of the compounds against ATM in vitro, the following assay was used to determine IC₅₀ values.

ATM protein was immunoprecipitated from HeLa cell nuclear extract using rabbit polyclonal anti-sera raised to the C-terminal ˜500 amino-acid residues of the human ATM protein. The immunoprecipitation was performed according to the methodology described by Banin, S. et al. (1998). 10 μl of immunoprecipitated ATM in Buffer C (50 mM Hepes, pH 7.4, 6 mM MgCl₂, 150 mM NaCl, 0.1 mM sodium orthovanadate, 4 mM MnCl2, 0.1 mM dithiothreitol, 10% glycerol) was added to 32.5 μl of buffer C containing 1 μg of the ATM substrate GSTp53N66 in a V-bottomed 96 well polypropylene plate. The GSTp53N66 substrate is the amino terminal 66 amino acid residues of human wild type p53 fused to glutathione S-transferase. ATM phosphorylates p53 on the residue serine 15 (Banin, S. et al. (1998)). Varying concentrations of inhibitor were then added. All compounds were diluted in DMSO to give a final assay concentration of between 100 μM and 1 nM, with DMSO being at a final concentration of 1%. After 10 minutes of incubation at 37° C., the reactions were initiated by the addition of 5 μl of 500 μM Na-ATP. After 1 hour with shaking at 37° C., 150 μl of phosphate buffered saline (PBS) was added to the reaction and the plate centrifuged at 1500 rpm for 10 minutes. 5 μl of the reaction was then transferred to a 96 well opaque white plate containing 45 μl of PBS to allow the GSTp53N66 substrate to bind to the plate wells. The plate was covered and incubated at room temperature for 1 hour with shaking before discarding the contents. The plate wells were washed twice by the addition of PBS prior to the addition of 3% (w/v) bovine serum albumin (BSA) in PBS. The plate was incubated at room temperature for 1 hour with shaking before discarding the contents and washing twice with PBS. To the wells, 50 μl of a 1:10,000 dilution of primary phosphoserine-15 antibody (Cell Signaling Technology, #9284L) in 3% BSA/PBS was added to detect the phosphorylation event on the serine 15 residue of p53 elicited by the ATM kinase. After 1 hour of incubation at room temperature with shaking, the wells were washed four times with PBS prior to the addition of an anti-rabbit HRP conjugated secondary antibody (Pierce, 31462) with shaking for 1 hour at room temperature. The wells were then washed four times with PBS before the addition of chemiluminescence reagent (NEN Renaissance, NEL105). The plate was then shaken briefly, covered with a transparent plate seal and transferred to a TopCount NXT for chemiluminescent counting. Counts per second, following a one second counting time, were recorded for each reaction.

The enzyme activity for each compound is then calculated using the following equation:

$$\% Inhibition = 100 - ( \frac{( \begin{matrix}cpm of unknown - \\ mean negative cpm\end{matrix} ) \times 100}{( \begin{matrix}mean positive cpm - \\ mean negative cpm\end{matrix} )} )$$

Compound (Ia) was assayed for ATM inhibition activity using the method described above and found to have an IC₅₀ value (the concentration at which 50% of the enzyme activity is inhibited) of less than 200 nM. This indicates that compound (Ia) inhibits ATM activity in vitro.

### Example 5

### Sensitisation of Cells to Ionising Radiation and Chemotherapeutics, and Repression of Viral Infection

To test the ability of compound (Ia) to sensitise cells to ionising radiation clonogenic survival assays were performed using the HeLa human tumour derived cell lines. Enough cells to give ˜100 colonies per treatment were seeded into 6 well dishes 4-6 hours prior to the addition of compound (Ia) at the concentrations shown in the table below. After 1 hour of incubation with the compound, cells were irradiated at 2 Gy/min using a Faxitron 43855D X-ray cabinet. For all treatments, after a further 16 hours incubation, drug containing media was removed and fresh media added prior to a further incubation of 10 days before the staining of colonies with Giemsa. All compounds were solubilised in DMSO, with a final concentration on cells of no more than 0.1%. Resulting colonies containing >50 cells were counted as positives.

The results are expressed as a sensitisation enhancement ratio (SER), a measure of sensitisation of cells to the effects of ionisation radiation. The SER is given by the following formula:

$$SER = \frac{cell death after irradiation in the presence of compound}{cell death after irradiation without compound}$$

Therefore an SER greater than 1.0 indicates enhancement of ionisation radiation-induced cell death. The table below shows the SER values obtained at the indicated concentrations of compound (Ia).

SER (HeLa cells; 2Gy irradiation)

| | |
|---|---|
| 100 nM compound (Ia) | 1.3 |
| 300 nM compound (Ia) | 3.0 |
| 500 nM compound (Ia) | 3.3 |

The table shows that compound (Ia) potentiates the cytotoxicity of ionising radiation on cells.

Compound (Ia) has also been found to potentiate the cytotoxic effects of etoposide, camptothecin and doxorubicin in vitro in clonogenic survival assays. This indicates that compound (Ia) is also suitable for use in combination with known chemotherapeutics.

The ability of compound (Ia) to repress retroviral infections was assessed using an HIV-based LUCIA assay. The LUCIA assay was carried out as described in WO 03/070726 (see pages 125-126 therein), which is incorporated herein by reference. Compound (Ia) was found to repress retroviral infection with an IC₅₀ of 1.3 μM.

### Example 6

### Oral Bioavailability: Comparison with Related Compounds

The oral bioavailability of compound (Ia) was assessed by comparison with three structurally similar compounds (compounds (II)-(IV)).

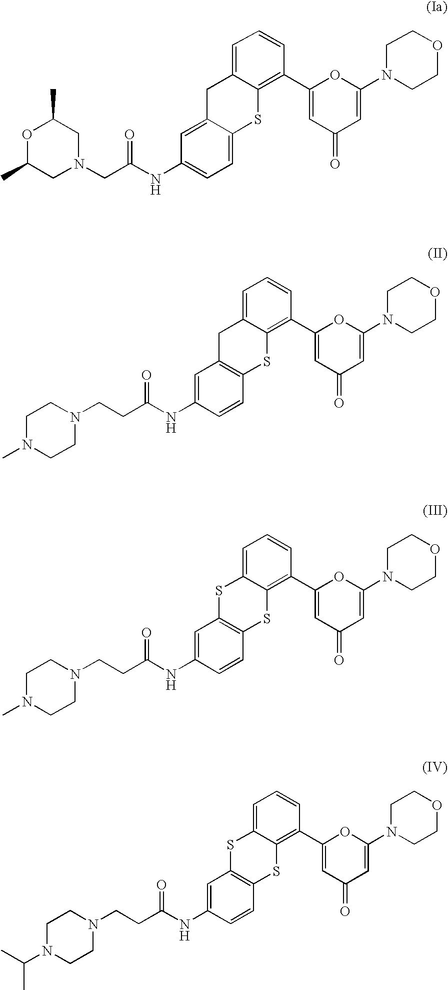

The compounds (II)-(IV) are all ATM inhibitors and have been described in the previous PCT applications WO 03/070726 and WO 2005/016919 (both incorporated herein by reference). The compounds (II)-(IV) were selected for comparison with compound (Ia) from the broad class of ATM inhibitors previously described because they exhibit a high degree of structural similarity to compound (Ia).

The synthesis of compound (II) is described in WO 03/070726 (identified therein as compound 71), which is incorporated herein by reference. The synthesis of compounds (III) and (IV) is described in WO 2005/016919 (identified therein as compounds 94 and 118, respectively), which is incorporated herein by reference.

In order to assess the oral bioavailability of compounds (Ia)-(IV), the compounds were dissolved to 5 mg/ml in an aqueous formulation which was then dosed orally and intravenously at 10 mg/kg to discrete groups of mice. For the intravenously dosed animals, blood samples were taken (three mice per time point) at 3, 5, 15, 30, 60, 120, 240 and 360 minutes post-dose. For the orally dosed animals, blood samples were taken (three mice per time point) at 5, 15, 30, 60, 120, 240 and 360 minutes post-dose. The plasma concentration of each compound at all time points, for both dose routes, was determined by HPLC-MS/MS analysis. The details of the HPLC-MS/MS method are as follows.

Sample was extracted for analysis by homogenisation of tissue, if required, vortex mixing and then centrifugation to produce a compound-containing supernatant that can be assayed by HPLC-MS/MS. Quantitation was achieved by comparison to a serial dilution of a known amount of parent compound. The chromatographic and MS conditions used are given below.

| LC Column | Synergyi MAX-RP 50 × 2 mm (Phenomenex) | | |
|---|---|---|---|
| Mobile phase | A Acetonitrile | | |
| | B Formic acid (0.01%) | | |
| Mobile phase | 50:50 v/v Acetonitrile:Formic acid (0.01%) | | |
| (isocratic pump) | | | |
| Flow rate | 700 μl/min | | |
| Gradient | Time | A (%) | B (%) |
| | 0 | 5 | 95 |
| | 1.0 | 5 | 95 |
| | 5.0 | 95 | 5 |
| | 5.1 | 5 | 95 |
| | 6.5 | 5 | 95 |

| | |
|---|---|
| Run time | approximately 7 mins |
| Needle wash | Acetonitrile |

MS conditions were determined by infusing compound dissolved in 50:50 acetonitrile:formic acid (0.01%).

Generic gas settings and TIS voltage were then used.

Curtain gas 40 eV

TIS 5000V

Gas 1 30

Gas 2 70

Collision gas 6

Temperature 500

Total exposure as indicated by area under the curve (AUC) was calculated. By definition, intravenous delivery results in 100% bioavailability and the comparative bioavailability obtained through oral delivery is calculated by:

$$\frac{Dose \times {AUC}_{oral}}{Dose \times {AUC}_{iv}} \times 100 = \% F$$

The results are expressed as percentages shown below.

| Compound (Ia) | Compound (II) | Compound (III) | Compound (IV) |
|---|---|---|---|
| 37% | 0% | 0% | 0% |

As can be seen from the above table, compound (Ia) was the only ATM inhibitor of those tested which was found to be orally bioavailable.

### Example 7

### Tissue Pharmacokinetics: Comparison with Related Compounds

The tissue distribution of compound (Ia) was assessed by comparison with the same three structurally related analogues used in Example 6 (compounds (II)-(IV)).

In order to assess tissue pharmacokinetics, a single intraperitoneal (i.p.) dose of 50 mg/kg of each compound was administered to discrete groups of mice. Blood and tumour samples were taken (three mice per time point) at 1 and 4 hours post-dose. The concentration of each compound in both matrices at both time points was determined by HPLC-MS/MS analysis using the method described above for Example 6. The mean concentrations found are presented below.

| | | Compound | Compound | Compound | Compound |
|---|---|---|---|---|---|
| 50 mg/kg i.p. | | (Ia) | (II) | (III) | (IV) |
| Plasma | 1 hr | 31766 | 3026 | 2032 | 1916 |
| (ng/ml) | 4 hr | 5963 | 137 | 626 | 383 |
| Tumour | 1 hr | 19733 | 2807 | 1730 | 899 |
| (ng/g) | 4 hr | 4143 | 1060 | 2191 | 1090 |

It is clear that compound (Ia) achieved the highest plasma and tumour concentrations at both time points.

### Example 8

### Maximum Tolerable Dose: Comparison with Related Compounds

The in vivo toxicity of compound (Ia) was assessed by comparison with the same three structurally related analogues used in Examples 6 and 7 (compounds (II)-(IV)).

The maximum single tolerable intravenous dose which could be administered to rats and mice was determined. Escalating doses of each compound were given to discrete, naive pairs of animals (1 male and 1 female) until a dose that elicited clinical signs, was reached. Then for each compound, two further animals were then treated with the highest dose that had not produced any clinical signs. The highest dose of each compound that did not elicit clinical signs in 4 treated animals was deemed to be the maximum tolerated dose (MTD). MTD values are shown below.

| | Compound | Compound | Compound | Compound |
|---|---|---|---|---|
| | (Ia) | (II) | (III) | (IV) |
| Rat MTD | >100 mg/kg | 40 mg/kg | 75 mg/kg | Not |
| (i.v.) | | | | tested |
| Mouse MTD | 75 mg/kg | <40 mg/kg | <40 mg/kg | <40 mg/kg |
| (i.v.) | | | | |

These results show that compound (Ia) generally exhibited the highest MTD, indicating that compound (Ia) shows lower overall toxicity to an organism than structurally related compounds.

### Example 9

### Measuring Effect of Compound (Ia) on Cells which Differ as to their Mismatch Repair Status

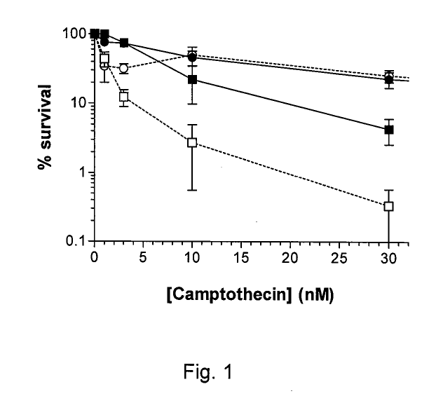

The effect of ATM inhibition on HCT116 and HCT116 (+chromosome 3) cellular survival in response to the topoisomerase I inhibitor camptothecin was measured by clonogenic assays. Tissue culture treated 6-well plates were seeded at an appropriate concentration to give 100-200 colonies per well and returned to the incubator in order to allow the cells to attach. Four hours later, the compound (Ia)(1 micromolar) or vehicle control (0.1% DMSO) was added to the cells. The cells were incubated for 1 hour in the presence of inhibitor prior to the addition of camptothecin. The cells were then incubated for 16 hours before the media was replaced with fresh DMEM in the absence of drugs. After ˜10 days colonies formed were fixed and stained with Giemsa (Sigma, Poole, UK) and scored using a ColCount automated colony counter (Oxford Optronics Ltd, Oxford, UK). The data was calculated as surviving fractions with respect to vehicle controls±SE. The results are shown in FIG. 1, in which the results are shown as HCT116+Chr3 (MMR+): with vehicle only ●, with vehicle and 1 μM of compound (Ia) ∘; HCT116 (MMR−): with vehicle only ▪, with vehicle and 1 μM of compound (Ia) □.

1. A compound of formula (I): 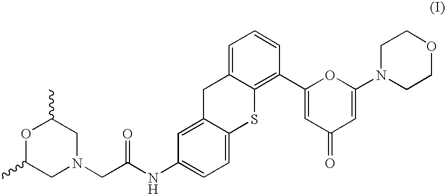
and isomers, salts, solvates, chemically protected forms, and prodrugs thereof.

2. A compound according to claim 1 of formula (Ia):

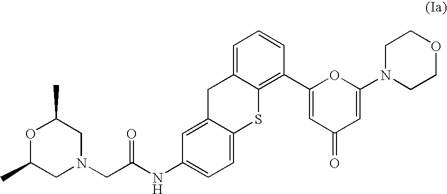

3. A compound according to claim 1 of formula (Ib):

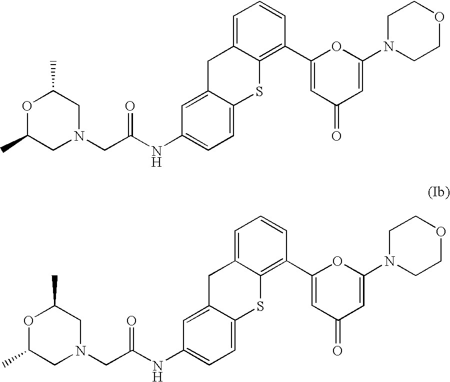

4. A composition comprising a compound according to any one of claims 1 to 3 or a pharmaceutically acceptable salt thereof and a pharmaceutically acceptable carrier or diluent.

5. A method of treatment of a mammal comprising administering a therapeutically effective amount of a compound according to any one of claims 1 to 3 or a pharmaceutically acceptable salt thereof to a mammal in need thereof.

6. The method according to claim 5 wherein the mammal is in need of inhibition of ATM activity.

7. The method according to claim 5 wherein the mammal is in need of cancer therapy.

8. The method according to claim 5 wherein the mammal is in need of antiretroviral therapy.

9. The method according to claim 5 wherein the mammal is human.

10. A method of synthesising a compound as defined in claim 1, comprising reacting 2-(7-amino-9H-thioxanthen-4-yl)-6-morpholin-4-yl-pyran-4-one with chloracetyl chloride followed by 2,6-dimethylmorpholine.
